(19) United States
(12) Patent Application Publication
(10) Pub. No.: US 2013/0139289 A1
(43) Pub. Date: Jun. 6, 2013

(54) Headgear with Retractable Shade

(71) Applicant: Adrianne Booth Jackson, Monticello, NY (US)

(72) Inventor: Adrianne Booth Jackson, Monticello, NY (US)

(21) Appl. No.: 13/705,483

(22) Filed: Dec. 5, 2012

Related U.S. Application Data
(60) Provisional application No. 61/566,757, filed on Dec. 5, 2011.

(51) Int. Cl. A61F 9/04

(52) U.S. Cl. CPC A61F 9/045
USPC 2/12

(57) ABSTRACT

A sleep curtain device comprised of an expandable headband for placement on a wearer's head and at least one shade repositionably attached to said headband to allow the wearer to reposition the shade 360 degrees around the wearer's head without removing the device from the wearer's head. The sleep curtain device has many uses including, without limitation, shielding a wearer's face from public view and/or unwanted light, such as sunlight and ambient light.

### CROSS-REFERENCE

This application claims priority from Provisional Patent Application Ser. No. 61/566,757 filed on Dec. 5, 2012.

### FIELD OF THE INVENTION

This invention relates to a headgear device with a retractable shade for covering a wearer's face and/or blocking out light, such as sunlight and/or ambient light, as well as harmful ultraviolet rays.

### BACKGROUND

Many passengers enjoy sleeping in a car, bus, train and/or airplane as a means of passing time and/or catching up on their sleep while travelling. Similarly, many beachgoers and other sunbathers enjoy resting on a beach or in the warmth of the sun. However, when sleeping sitting up or in a reclined position, an individual's mouth will tend to open and/or the individual will begin to drool from their mouth, both of which conditions are unsightly and lead to embarrassment for the dozing individual when he or she awakens. Additionally, as the individual is sleeping, his or her head may fall to one side or another or light, such as sunlight, may fall upon the passenger's face and prevent the individual from falling asleep or achieving a restful sleep.

In an effort to overcome the above described difficulties, an individual may attempt to cover his or her face with a blanket or a towel to prevent other passengers from seeing the individual in a state of sleep and/or to reduce or eliminate sunlight and/or ambient light from preventing the individual from falling asleep or achieving a restful sleep. However, these types of devices tend to be ineffective because they are not securely attached to the wearer. More specifically, the blanket or towel may fall off of the wearer's face as the wearer falls asleep or repositions his or her head, thereby exposing the wearer's face to the other passengers, which can be embarrassing to the wearer and may discourage the individual from getting much needed rest. Additionally, the absence of the towel or blanket may permit light, such as sunlight or ambient light, to fall upon the wearer's face, thereby prematurely awakening the wearer from his or her sleep. Finally, an unkempt blanket or towel on a user's head may be unsightly or appear disheveled, which is also undesirable.

Consequently, there exists in the art a long-felt need for a sleep curtain or headgear device with a retractable shade for covering a wearer's face and/or blocking out light, such as sunlight or ambient light, as well as harmful ultraviolet rays as the wearer sleeps. There also exists in the art a long felt need for a headgear device with a retractable shade portion that can not only be deployed on demand to cover a wearer's face, but that can also function to protect the back of the wearer's neck from unwanted sunlight and/or ultraviolet rays when in a stowed or partially stowed position without removing the device from the wearer's head. Finally, there is a long-felt need for a headgear device with a retractable shade portion that accomplishes all of the forgoing objectives and that is relatively inexpensive to manufacture, aesthetically pleasing, and safe and easy to use.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, is a device comprised of a headband and at least one, and preferably two, retractable shades repositionally attached thereto. The headband portion is preferably comprised of a continuous loop of elastic material with a continuous opening therein for receipt of a wearer's head. The retractable shade portion is preferably comprised of an opaque and sun-blocking or sun reducing fabric or other suitable material that is repositionably attached to said headband and repositionable between a deployed position and a stowed position. In the deployed position, the retractable shade portion hangs downwardly from the headband and over at least a portion of the wearer's face, thereby covering the same from public view and preventing or reducing unwanted light, such as sunlight and/or ultraviolet rays, from falling upon the wearer's face. In the stowed or partially stowed position, the retractable shade portion hangs downwardly from the headband and over at least a portion of the sides of wearer's face and/or the back of the neck, thereby covering the same from view and from unwanted light, such as sunlight and/or ultraviolet rays. When in the stowed position, the retractable shade portion does not substantially cover the wearer's face.

The sleep curtain device of the present invention allows the wearer to reposition the shade 360 degrees around the wearer's head without removing the device from the wearer's head. The sleep curtain device has many uses including, without limitation, shielding a wearer's face from public view and/or unwanted light, such as sunlight and ambient light, as well as harmful ultraviolet rays.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

FIG. 1 illustrates a perspective view of a preferred embodiment of the sleep curtain device of the present invention.

FIG. 2 illustrates a perspective view of a preferred embodiment of the sleep curtain device of the present invention in a deployed position on a wearer's head.

FIG. 3 illustrates a perspective view of a preferred embodiment of the sleep curtain device of the present invention in a stowed position on a wearer's head.

FIG. 4 illustrates a top perspective view of a preferred embodiment of the sleep curtain device of the present invention.

FIG. 5 illustrates an alternate embodiment of the present invention with a single shade installed thereon.

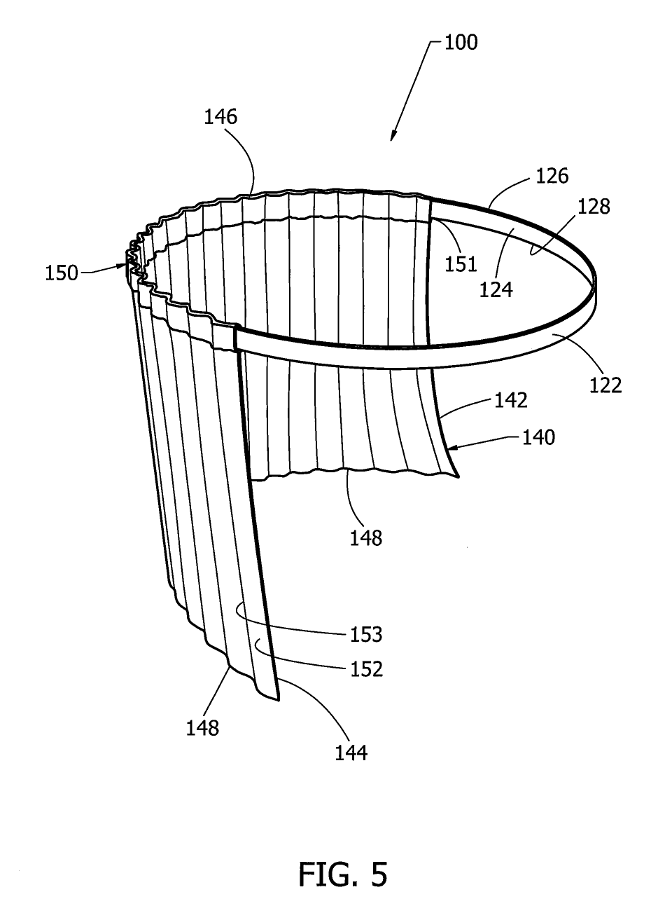

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details.

The sleep curtain device of the present invention allows the wearer to reposition the shade 360 degrees around the wearer's head without removing the device from the wearer's head, and the shade enables the wearer to shield his or her face from public view and/or unwanted light, such as sunlight and ambient light, as well harmful ultraviolet rays. Additionally, the headband portion of the device is comprised of an elastic material that is expandable to fit a wearer's head regardless of shape and/or size. Finally, the sleep curtain device of the present invention is relatively inexpensive to manufacture, aesthetically pleasing and safe and easy to use.

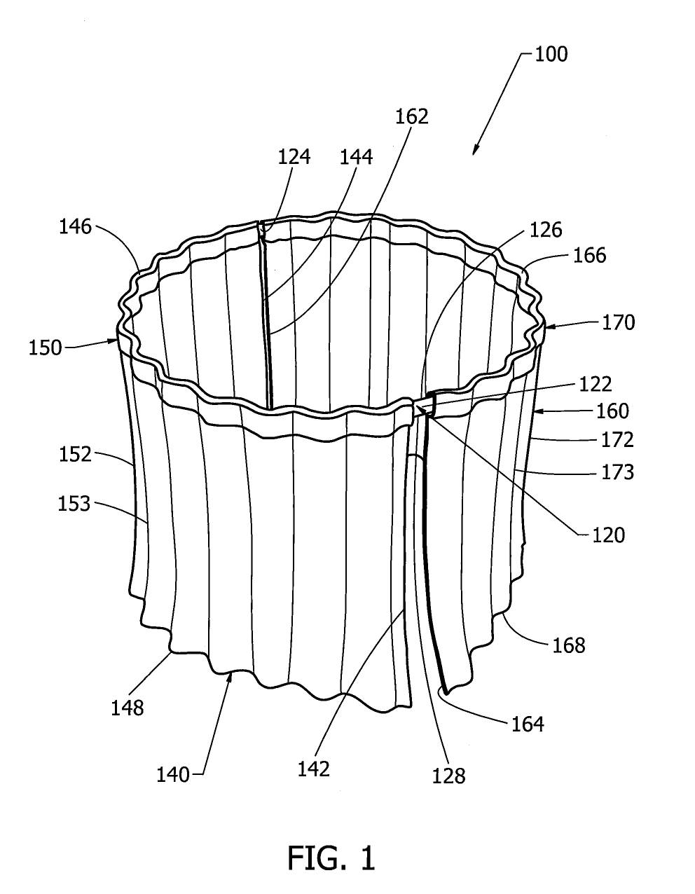

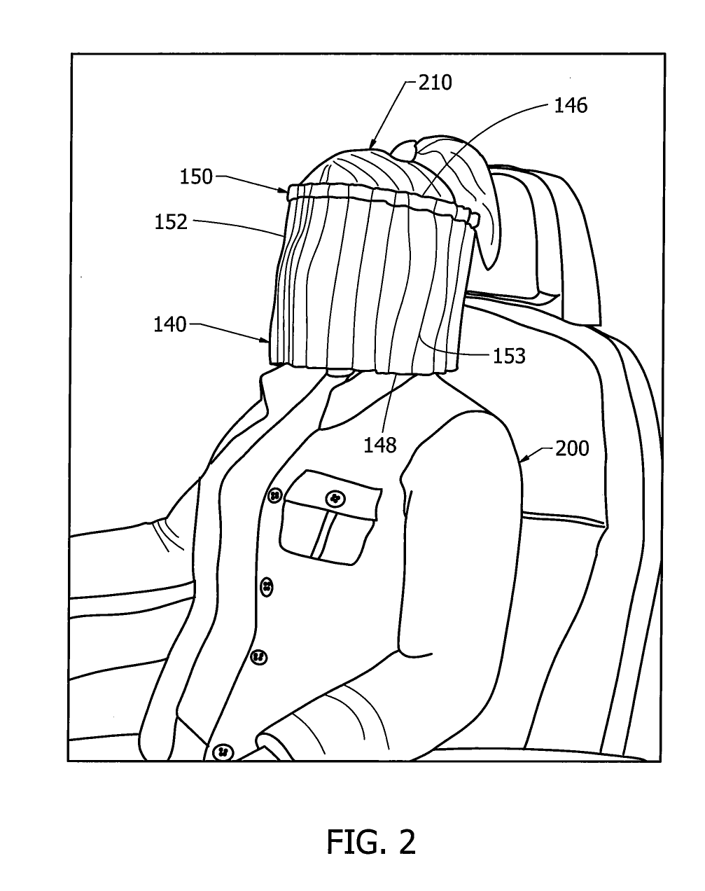

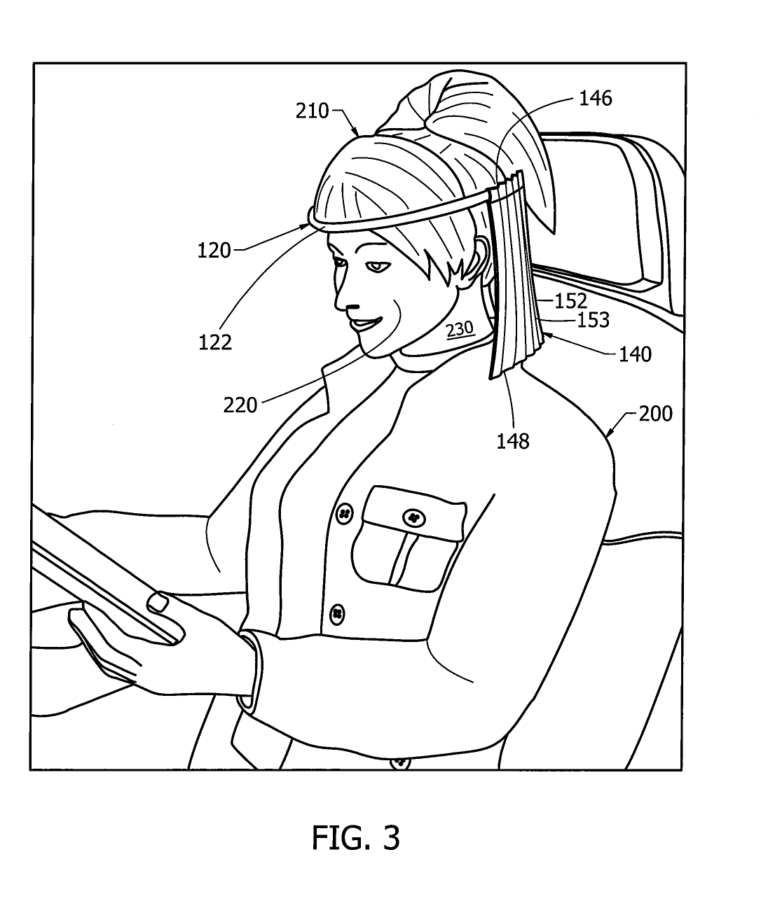

Referring initially to the drawings, FIG. 1 illustrates a perspective view of a preferred embodiment of the sleep curtain device 100 of the present invention, and FIGS. 2 and 3 illustrate a perspective view of the device 100 installed on a user's head 210 in a deployed and stowed position, respectively. Device 100 is preferably comprised of a headband 120, a first shade 140 and a second shade 160. Headband 120 is preferably comprised of a continuous loop of expandable material such as elastic or covered elastic so that headband 120 is capable of expanding and/or conforming to heads of various shapes and sizes. The general elasticity of headband 120 permits the headband to expand and conform to various head shapes and sizes. Notwithstanding, it is also contemplated that headband 120 could be comprised of other suitable materials such as plastic, vinyl, lace, niche, bejeweled, etc., without affecting the overall concept of the present invention.

Headband 120 is further comprised of an outer surface 122 that faces outwardly from the user's head 210, an inner surface 124 substantially in contact with the user's head 210, a top portion 126, a bottom portion 128 and a continuous opening 130 therein for receipt of a user's head 210 as shown in FIGS. 2 and 3. In its unexpanded form, headband 120 is preferably between 16 and 20 inches in overall length or circumference, between ⅛th and ½ of an inch in width as measured from top portion 126 to bottom portion 128 and between 1/32 and ⅛ of an inch in thickness as measured from outer surface 122 to inner surface 124, though it is contemplated that other sizes of headband 120 could also be used without affecting the overall concept of the present invention.

As stated above, device 100 further comprises at least one and preferably two shades 140, 160. Prior to installation on headband 120, first shade 140 is preferably a generally rectangular drape-like covering comprised of a first end 142, a second end 144, a top 146 and a bottom 148. First shade 140 further comprises a loop portion 150 that extends from first end 142 to second end 144 near top 146, and a curtain 152 that extends downwardly from said loop portion 150 to bottom 148 and from first end 142 to said second end 144. Loop portion 150 further comprises a continuous opening 151 therein that also extends from first end 142 to second end 144 and is for receipt of headband 120, as described more fully below.

Loop portion 150 and curtain 152 may be of unitary construction, or may be separate units attached together by any common means known in the art just as stitching, etc. In preferred embodiment of the present invention, first shade 140 is preferably comprised of a generally opaque material, such as lace with an opaque backing, cotton, polyester, garbadine, silk or any other lightweight fabric that is also capable of blocking or reducing the amount of ultraviolet rays that pass through first shade 140.

First shade 140 is preferably between 8 and 16 inches in length, as measured from top 146 to bottom 148, and preferably between four and ten inches in width, as measured from first end 142 to second end 144 when extended, though other sizes may be used to suit user preference. First shade 140 may also come in various colors or prints to suit user preference and/or, for example, to match a particular outfit of the user. As shown in the FIGS., curtain 152 may also comprise a number of creases or folds 153 to give first shade 140 the appearance of drapery. Not only do creases 153 add ornamental flair to device 100, but they also permit first shade 140 to be more compactly stowed when not in use as shown in FIG. 3 inasmuch as first shade 140 can be folded accordion style.

As indicated above, first shade 140 is repositionably attached to headband 120 by inserting headband 120 in opening 151 of loop portion 150. Once installed, first shade 140 can be positioned anywhere along headband 120 by wearer 200, thereby providing wearer 200 with the ability to position and/or reposition the shade anywhere (i.e., 360 degrees) around the wearer's head 210 without removing device 100 from wearer's head 210. For example, wearer 200 may elect to position first shade 140 in a deployed position as shown in FIG. 2 to conceal his or her face from public view and/or to block out unwanted light so that the wearer can rest, or in a stowed position as shown in FIG. 3, meaning that first shade 140 is withdrawn from the face area and a substantial portion of the wearer's face 220 is exposed. By enabling wearer 200 to reposition first shade 140 between a deployed and a stowed position, and anywhere in between, wearer 200 has the ability to shield his or her face from public view and/or unwanted light, such as sunlight and/or ambient light, as well as harmful ultraviolet rays, on demand and without the need to remove device 100 from wearer's head 210.

As stated above, device 100 preferably also comprises a second shade 160. Prior to installation on headband 120 and similar to the construction of first shade 140, second shade 160 is also preferably a generally rectangular drape-like covering comprised of a first end 162, a second end 164, a top 166 and a bottom 168. Second shade 160 further comprises a loop portion 170 that extends from first end 162 to second end 164 near top 166, and a curtain 162 that extends downwardly from said loop portion 170 to bottom 168 and from first end 162 to second end 164. Loop portion 170 further comprises a continuous opening 171 therein that also extends from first end 162 to second end 164 and is for receipt of headband 120 in the same manner described above with respect to opening 151 of loop portion 150 of first shade 140.

Similar to first shade 140, loop portion 170 and curtain 172 may also be of unitary construction, or may be separate units attached together by any common means known in the art just as stitching, etc. In preferred embodiment of the present invention, second shade 160 is preferably comprised of a generally opaque material, such as lace with an opaque backing, cotton, polyester, garbadine, silk or any other lightweight fabric that is also capable of blocking or reducing the amount of ultraviolet rays that pass through second shade 160.

Second shade 160 is preferably sized to match the dimensions of first shade 140 for aesthetic reasons and is between 8 and 16 inches in length, as measured from the top 166 to bottom 168, and preferably between four and ten in width, as measured from first end 162 to second end 164, though other sizes may be used to suit user preference without affecting the overall concept of the present invention. For example, the length of second shade 160 may be longer than that of first shade 140, or vice versa, to extend further down the wearer's neck 230, thereby providing additional protection from harmful ultraviolet rays. Second shade 160 may also come in various colors or prints to suit user preference and/or to match a particular outfit of the user or to match first shade 140. As shown in the FIGS., curtain 172 may also comprise a number of creases or folds 153 to give second shade 160 the appearance of drapery. Not only do creases 173 add ornamental flair to device 100, but they also permit second shade 160 to be more compactly stowed when not in use, as described above with respect to first shade 140.

As indicated above, second shade 160 is also repositionably attached to headband 120 by inserting headband 120 into opening 171 of loop portion 170. Once installed, second shade 160 can be positioned or repositioned anywhere along headband 120 by wearer 200, thereby providing wearer 200 with the ability to position and/or reposition second shade 160 anywhere (i.e., 360 degrees) around the wearer's head 210 without removing device 100 from wearer's head 210. For example, wearer 200 may elect to position second shade 160 in a deployed position (as defined above) to conceal his or her face from public view and/or to block out unwanted light so that wearer 200 can rest, or in a stowed position, meaning that second shade 160 is withdrawn from the face area and a substantial portion of the wearer's face 220 is exposed. By enabling wearer 200 to reposition second shade 160 between a deployed and a stowed position, and anywhere in between, wearer 200 has the ability to shield his or her face from public view and/or unwanted light, such as sunlight and/or ambient light, as well as harmful ultraviolet rays, on demand and without the need to remove device 100 from wearer's head 210.

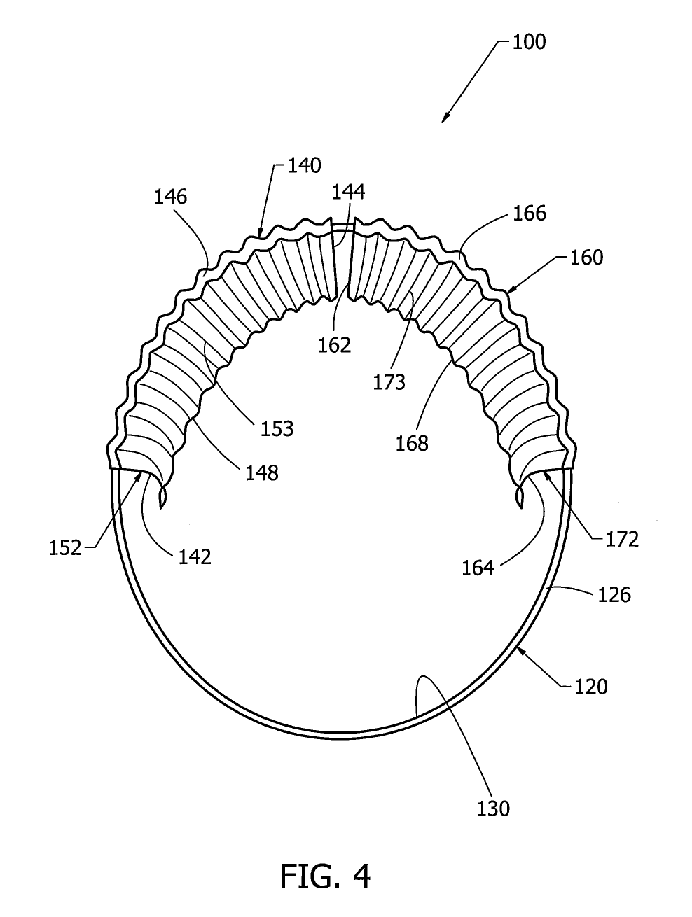

Additionally, it should be appreciated that first shade 140 and second shade 160 can be deployed in tandem and substantially adjacent to one another as shown in FIG. 4 to provide even further protection to the wearer. In fact, first shade 140 and second shade 160 can be deployed to provide approximately 360 degrees of protection and/or coverage to a user, as shown in FIG. 1.

Consequently, the sleep curtain device 100 of the present invention allows the wearer to reposition the shade 360 degrees around the wearer's head without removing the device from the wearer's head, and the shade enables the wearer to shield his or her face from public view and/or unwanted light, such as sunlight and ambient light. The ability to conceal one's face from public view will encourage individuals who desire to nap in public to do so without embarrassment. Additionally, the headband portion of the device is comprised of an elastic material that is expandable to fit a wearer's head regardless of shape and/or size. The sleep curtain device of the present invention may be manufactured in a variety of colors and materials to suit user preference and/or appeal to a particular style of fashion. Finally, the sleep curtain device of the present invention is relatively inexpensive to manufacture, and safe and easy to use.

Additionally, other variations are within the spirit of the present invention. Thus, while the invention is susceptible to various modifications and alternative constructions, a certain illustrated embodiment thereof is shown in the drawings and has been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A sleep curtain device comprising:
a headband; and
a shade repositionably attached to said headband for covering a portion of a wearer's face.

2. The sleep curtain device of claim 1 wherein said shade is repositionable between a deployed position and a stowed position without removing the headband from a wearer's head.

3. The sleep curtain device of claim 1 wherein said headband is expandable in size.

4. The sleep curtain device of claim 2 wherein the shade covers a substantial portion of a wearer's face when the shade is in the deployed position.

5. The sleep curtain device of claim 1 wherein a substantial portion of a wearer's face is exposed when the shade is in the stowed position.

6. The sleep curtain device of claim 1 wherein the shade is comprised of at least one of an ultraviolet light blocking material and an opaque material.

7. A sleep curtain device comprising:
an expandable headband;
a first shade; and
a second shade, wherein said first shade and second shade are each repositionably attached to said headband and are each comprised of an ultraviolet light blocking material.

8. The sleep curtain device of claim 7 wherein each of said first shade and second shade is repositionable between a deployed position and a stowed position without removing the expandable headband from a wearer's head.

9. The sleep curtain device of claim 8 wherein the first shade and second shade combine to cover a substantial portion of a wearer's face when each of the first shade and second shade are in the deployed position.

10. The sleep curtain device of claim 8 wherein a substantial portion of a wearer's face is exposed when each of the first shade and the second shade is in the stowed position.

11. The sleep curtain device of claim 7 wherein at least one of said first and second shades is further comprised of an opaque material.

12. The sleep curtain device of claim 7 wherein said ultraviolet light blocking material is comprised of at least one of the following: a lace with a solid backing, a cotton, a polyester, a garbadine, or a silk.

13. The sleep curtain device of claim 11 wherein said opaque material is comprised of at least one of the following: a lace with a solid backing, a cotton, a polyester, a garbadine, or a silk.

14. A sleep curtain device comprising:
an expandable headband, wherein said expandable headband is comprised of a continuous loop of elastic material;
a first shade; and
a second shade, wherein each of said first shade and said second shade is comprised of a generally opaque material.

15. The sleep curtain device of claim 14 wherein each of said first shade and second shade is repositionable between a deployed position and a stowed position without removing the expandable headband from a wearer's head.

16. The sleep curtain device of claim 15 wherein the first shade and second shade combine to cover a substantial portion of a wearer's face when each of the first shade and second shade are in the deployed position and adjacent to one another.

17. The sleep curtain device of claim 15 wherein a substantial portion of a wearer's face is exposed when each of the first shade and the second shade is in the stowed position.

18. The sleep curtain device of claim 14 wherein at least one of said first and second shades is further comprised of an ultraviolet light blocking material.

19. The sleep curtain device of claim 18 wherein said ultraviolet light blocking material is comprised of at least one of the following: a lace with a solid backing, a cotton, a polyester, a garbadine, or a silk.

20. The sleep curtain device of claim 14 wherein said generally opaque material is comprised of at least one of the following: a lace with a solid backing, a cotton, a polyester, a garbadine, or a silk.
